SEQUENCE LISTING (2 sequences)

SEQ ID NO: 1 - protein, 572 aa, Homo Sapiens
MYRSGERLLGSHALPAEQRDFLPLETTNNNNNHHQPGAWARRAGSSASSPPSASSSPHPSAAVPAADPADSASGSSNKRKRDNKASGGRAAGGGRADGGGVVYSGTPWKRRNYNQGVVGLHEEISDFYEYMSPRPEEEKMRMEVVNRIESVIKELWPSADVQIFGSFKTGLYLPTSDIDLVVFGKWENLPLWTLEEALRKHKVADEDSVKVLDKATVPIIKLTDSFTEVKVDISFNVQNGVRAADLIKDFTKKYPVLPYLVLVLKQFLLQRDLNEVFTGGIGSYSLFLMAVSFLQLHPREDACIPNTNYGVLLIEFFELYGRHFNYLKTGIRIKDGGSYVAKDEVQKNMLDGYRPSMLYIEDPLQPGNDVGRSSYGAMQVKQAFDYAYVVLSHAVSPIAKYYPNNETESILGRIIRVTDEVATYRDWISKQWGLKNRPEPSCNGPVSSSSATQSSSSDVDSDATPCKTPKQLLCRPSTGNRVGSQDVSLESSQAVGKMQSTQTTNTSNSTNKSQHGSARLFRSSSKGFQGTTQTSHGSLMTNKQHQGKSNNQYYHGKKRKHKRDAPLSDLCR

SEQ ID NO: 2 - DNA, 451 bp, Homo Sapiens
gggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgc

(19) United States
(12) Patent Application Publication
(10) Pub. No.: US 2022/0378761 A1
(43) Pub. Date: Dec. 1, 2022

(54) PAPD5 Inhibition As A Treatment For Dyskeratosis Congenita, Aplastic Anemia And Myelodysplastic Syndrome Caused By Reduced Telomerase RNA Levels

(71) Applicants: The Regents of The University of Colorado, A Body Corporate, Denver, CO (US); Washington University in St. Louis, St. Louis, MO (US)

(72) Inventors: Roy R. Parker, Boulder, CO (US); Siddharth Shukla, Boulder, CO (US); Luis Francisco Zirnberger Batista, St. Louis, MO (US); Hochang Jeong, St. Louis, MO (US)

(21) Appl. No.: 17/775,133

(22) PCT Filed: Nov. 5, 2020
(86) PCT No.: PCT/US2020/059089

§ 371 Date: May 6, 2022

Related U.S. Application Data
(60) Provisional application No. 62/932,191, filed on Nov. 7, 2019.

(51) Int. Cl. A61K 31/4375

(52) U.S. Cl. CPC A61K 31/4375

(57) ABSTRACT

The present invention includes the novel therapeutic strategies, systems., and compositions for the treatment of telomere-associated disease or disorder through the inhibition of PAPD5/7. the present invention includes the novel therapeutic application of PAPD5 inhibitors for the treatment of disease conditions that implicate reduced telomerase RNA levels. In particular, the present invention includes the novel therapeutic application of PAPD5 inhibitors for the treatment of dyskeratosis congenita, aplastic anemia, and myelodysplastic syndrome. In one preferred embodiment, the present invention includes the novel therapeutic application of RG7834 for the treatment of disease conditions that implicate reduced telomerase RNA levels. In particular, the present invention includes the novel therapeutic application of RG7834 for the treatment of dyskeratosis congenita, aplastic anemia, and myelodysplastic syndrome.

### CROSS-REFERENCE TO RELATED APPLICATIONS

This application claims the benefit of and priority to U.S. Provisional Application No. 62/932,191, filed Nov. 7, 2019, which is incorporated herein by reference in its entirety.

### GOVERNMENT INTEREST

This invention was made with government support under grant numbers GM045443 and HL137793 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Nov. 5, 2020, is named 90245-00421-Sequence-Listing-AF.txt” and is 5.88 Kbytes in size.

### TECHNICAL FIELD

The present invention is related the novel therapeutic strategies for the treatment of telomere-associated diseases or disorders through the inhibition of the PAPD5 enzyme. In particular, the present invention includes the novel therapeutic application of RG7834 for the treatment of telomere-associated diseases or disorders such as dyskeratosis congenita, aplastic anemia, and myelodysplastic syndrome.

### BACKGROUND

Dyskeratosis congenita (DC) is a pediatric bone marrow failure syndrome caused by germline mutations in telomere biology genes. DC) is characterized by a classic triad of dysplastic nails, lacy reticular pigmentation of the upper chest and neck, and oral leukoplakia. Patients with DC present at early age with short telomeres, and bone marrow failure represents the major cause of morbidity. Treatment is challenging and tailored to the individual. Hematopoietic stem cell transplantation remains the only curative treatment of bone marrow failure in these patients but historically has had poor long-term efficacy. Novel alternatives for clinical management are urgently needed.

Mutations in patients with DC are found in different genes involved in telomere protection or maintenance. Four of these genes impair the function of the telomerase RNA component (“TERC,” also referred to herein as “hTR”) leading to reduced telomerase activity, including mutations in TERC itself, poly(A)-specific ribonuclease (PARN), NAF1,10 and DKC1, which represents the most commonly mutated gene in DC (X-linked inheritance). Mutations in PARN and DKC1 reduce TERC stability and cause increased TERC degradation by the exosome component EXOSC10. Notably, degradation of TERC by the exosome can be inhibited by reducing the 39-end oligoadenylation of TERC through the modulation of PAPD5 [noncanonical poly(A) polymerase 5] levels. Utilizing the targeted differentiation of human embryonic stem cells (hESCs), the present investors showed that the genetic silencing of PAPD5 is able to improve the hematopoietic potential of DC cells thereby rescuing the major phenotype observed in this disease. This finding opens the possibility that the chemical inhibition of PAPD5 by a safe, efficient, orally available compound could represent a novel alternative to be pursued in the clinical management of patients with DC and mutations that impair TERC biology.

Here, the present inventors show that treatment with a novel PAPD5/7 inhibitor, RG7834, a novel small molecule inhibitor of PAPD5 and PAPD7, rescues TERC levels, restores correct telomerase localization in DKC1 and PARN-depleted cells, and is sufficient to elongate telomeres in DKC1_A353V hESCs. The present inventors also show that treatment with RG7834 significantly improved definitive hematopoietic potential from DKC1_A353V hESCs, indicating that the chemical inhibition of PAPD5, and in particular is a potential therapy for patients with DC and reduced TERC levels.

### SUMMARY OF THE INVENTION

As noted above, DC is a telomere biology disorder predominantly caused by impairment of hTR levels and function. Reduced hTR levels in DC patients leads to loss of telomerase function and premature shortening of telomeres, leading to bone marrow failure, widespread tissue dysfunction and mortality. Reduction in telomerase RNA degradation by inhibiting either the poly(A) polymerase PAPD5 or the 3′ to 5′ exonuclease EXOSC10 has been shown to rescue telomerase RNA levels and telomerase activity. More importantly, a partial depletion of PAPD5 is sufficient to rescue definitive hematopoietic differentiation of DKC1 mutant hESCs without indications of toxicity. Therefore, as noted above, PAPD5 is a potential therapeutic target for drug development for DC.

Here, the present inventors show that treatment of PARN- or DKC1-depleted cells with RG7834, a novel small molecule inhibitor of PAPD5 and PAPD7, leads to increased TERC levels and correct telomerase RNA subcellular localization. Moreover, RG7834 treatment significantly decreased the 39-end oligoadenylation of TERC in DKC1 A353V hESCs, leading to increased TERC levels in these cells. The chemical inhibition of PAPD5/7 was sufficient to elongate telomeres in DKC1 A353V hESCs and to significantly improve the definitive hematopoietic potential of these cells.

In one aspect, the present inventors sought to determine whether a small molecule inhibitor of PAPD5/7 called RG7834 inhibits TERC degradation and rescues TERC function. The present inventors treated DKC1 or PARN depleted HeLa cells with RG7834 and observed that RG7834 treatment rescued both TERC levels and localization to cajal bodies in DKC1 or PARN depleted HeLa cells. In this embodiment, RG7834 inhibits TERC degradation by preventing 3′ end oligoadenylation of TERC, which would otherwise lead to its degradation in a competitive manner by EXOSC10. The present inventors also observed that in a stem cell model of DC with a pathogenic DKC1 mutation, RG7834 treatment led to a rapid increase in telomere length treatment, reversing a key hallmark of DC. The increase in telomere length results from an increase in TERC levels without affecting TERT mRNA levels, suggesting that TERC is limiting for telomerase function in stem cells. The present inventors also observed that in a stem cell model of DC with a pathogenic DKC1 mutation, RG7834 treatment led to a rapid increase in telomere length after treatment, demonstrating that RG7834 treatment has long term efficacy in a DC model.

In one aspect, inhibition of the PAPD5 protein (SEQ ID NO. 1) may be employed as a therapeutic treatment for telomere biology diseases such as aplastic anemia, myelodysplastic syndrome, or familial pulmonary fibrosis, which are caused by reduced or disrupted TERC processing. Some examples of mutations in telomere maintenance genes that lead to these diseases include mutations in TERC, PARN and ZCCHC8. An important clinical criterion for the suitability of treatments that include the inhibition of PAPD5 may include mutations in telomere maintenance genes that reduce telomerase RNA accumulation in cells by disrupting its assembly into an RNP, but do not affect its function in telomere maintenance.

In another aspect, the specific PAPD5 inhibitor RG7834 may be a therapeutic candidate for the treatment of other telomere biology diseases such as aplastic anemia, myelodysplastic syndrome, or familial pulmonary fibrosis, which are caused by reduced or disrupted TERC processing. Some examples of mutations in telomere maintenance genes that lead to these diseases include mutations in TERC, PARN and ZCCHC8. An important clinical criterion for the suitability of RG7834 treatment may include mutations in telomere maintenance genes that reduce telomerase RNA accumulation in cells by disrupting its assembly into an RNP, but do not affect its function in telomere maintenance.

This disclosure therefore provides therapeutic strategies for the treatment of telomere-associated disease or disorder, such as DC, aplastic anemia, myelodysplastic syndrome, or familial pulmonary fibrosis, which may be caused by reduced or disrupted TERC processing, such treatment preferably in a human. In one preferred aspect, the invention may include a method for treating of a telomere-associated disease or disorder for which restoration of TERC processing may be beneficial.

In one aspect, the invention may include a method of treating a telomere-associated disease or disorder in a subject in need thereof, comprising administering a therapeutically effective amount of a PAPD5 inhibitor, which in a preferred embodiment may include the step of administering a therapeutically effective amount of RG7834, or a pharmaceutically acceptable salt thereof. In certain aspects, the invention may include a pharmaceutical composition for use in the treatment of a telomere-associated disease or disorder in a subject in need thereof, which may include a therapeutically effective amount of a PAPD5 inhibitor, such as of RG7834, or a pharmaceutically acceptable salt thereof and at least one pharmaceutically acceptable additive. In additional aspects, the invention may include a pharmaceutical kit for use in the treatment of a telomere-associated disease or disorder in a subject in need thereof containing a pharmaceutical composition including a therapeutically effective amount of a PAPD5 inhibitor, such as of RG7834, or a pharmaceutically acceptable salt thereof and at least one pharmaceutically acceptable additive, prescribing information for the composition, and a container. Additional aspects of the invention may further include a method of treating a telomere-associated disease or disorder in a subject in need thereof, comprising administering a therapeutically effective amount of a PAPD5 inhibitor, such as of RG7834, or a pharmaceutically acceptable salt thereof, wherein a therapeutically effective amount of a PAPD5 inhibitor may exhibit one or more of the following therapeutic actions:
rescue human telomerase RNA (TERC) function in the subject;
inhibit degradation of TERC in the subject;
increase levels of TERC in the subject;
increase telomerase activity in the subject;
increase telomere homeostasis in the subject;
modulate localization of TERC in the subject;
inhibit DNA damage signaling arising from eroded telomeres in the subject;
increase localization of TERC to the cajal bodies of the nucleus of one or more cells of the subject;
increase telomere length, and preferably increase telomere lengths in human embryonic stem cells (hESCs) carrying the DKC1_A353V mutation;
increase hematopoietic differentiation, and preferably hematopoietic potential in hESCs carrying the DKC1_A353V mutants;
increase telomere lengths in DKC1 depleted cells of the subject;
inhibit 3′ end oligoadenylation of TERC in the subject; and
increase telomere lengths in P ARN depleted cells of the subject.

Additional aspects of the invention may further include a method of treating a telomere-associated disease or disorder in a subject in need thereof, comprising administering a therapeutically effective amount of a PAPD5 inhibitor, such as of RG7834, or a pharmaceutically acceptable salt thereof, wherein a therapeutically effective amount of a PAPD5 inhibitor may prevent one or more of the following indications of said telomere-associate disease or condition:
loss of normal human telomerase RNA component (TERC) function in the subject;
atypical degradation of TERC in the subject;
decreased levels of TERC in the subject;
decreased telomerase activity in the subject;
decreased telomere homeostasis in the subject;
atypical localization of TERC in the subject;
increased DNA damage signaling arising from eroded telomeres in the subject;
decreased hematopoietic differentiation, and preferably hematopoietic potential in hESCs carrying the DKC1_A353V mutants;
decreased localization of TERC to the cajal bodies of the nucleus of one or more cells of the subject;
decreased telomere lengths, and in particular decreased telomere lengths in human embryonic stem cells (hESCs) carrying the DKC1_A353V mutation;
increased 3′ end oligoadenylation of TERC in the subject;
decreased telomere lengths in DKC1 depleted cells of the subject; and
decreased telomere lengths in PARN depleted cells of the subject.

Additional aims of the inventive technology will be evident from the detailed description and figures presented below.

### BRIEF DESCRIPTION OF DRAWINGS

FIG. 1. RG7834 treatment rescues TERC levels and localization in DKC1- and PARN-depleted cells. (A) Representative northern blots for TERC levels in HeLa cells under indicated conditions. Numbers below panels: average 6 standard deviation for 3 biological replicates. (B) Quantification of TERC by quantitative reverse transcription polymerase chain reaction in HeLa cells under the indicated conditions (n 5 3; biological replicates). Values are expressed in relation to scrambled control. (C) Representative images for 49,6-diamidino-2-phenylindole (DAPI) (nucleus), coilin (cajal body), TERC, and merge of individual channels obtained from HeLa cells transfected with indicated siRNAs and treated with RG7834. White arrows indicate TERC localization within the cell. Scale bar, 5 mm. Numbers in image panels: Quantification of fraction of cells with TERC colocalized to cajal bodies from at least 30 independent cells and 3 replicates. Cells with at least one TERC focus colocalized with coilin were counted. (D) Telomerase activity by telomere repeat amplification in HeLa cells transfected with indicated siRNAs and treated with 5 mM of RG7834 or DMSO. Range of protein concentrations represent fourfold serial dilutions. *P, 0.05; **P, 0.01. LC, loading control.

FIG. 2. RG7834 treatment rescues telomerase activity, increases telomere length, and improves hematopoietic specification in DKC1_A353V mutant hESCs. (A) Quantification of oligo(A) reads at the 3′ end (UGC) of TERC in indicated conditions (average±standard deviation from 2 independent replicates). TERC reads with more than 2 As at the 3′ end are considered oligoadenylated. (B) Quantification of TERC and TERT by quantitative reverse transcription polymerase chain reaction in DKC1_A353V hESCs treated with DMSO or different concentrations of RG7834 for 30 days (n=3; biological replicates). (C) Telornerase activity by telomere repeat amplification in DKC1_A353V hESCs treated with DMSO or different concentrations of RG7834. Range of protein concentrations represent fourfold serial dilutions. (D) Telomere length analysis by telomere restriction fragment analysis of DKC1_A353 V hESCs treated with DMSO or different concentrations of RG7834 for 90 days. (E) Representative flow cytometric analysis of CD34 and CD43 expression on day 8 of definitive hematopoietic differentiation, following CHIR99021 and SB-431542 treatment in DKC1 A353V cells treated with DMSO or different concentrations of RG7834, (F) Quantification of CD34⁺ CD43⁻ population obtained from day 8 differentiation cultures treated with CHIR99021 and SB-431542, as in panel E. (G) CFC potential of definitive hematopoietic progenitors in WT and DKC1_A353V cells treated with DMSO or 1 μM of RG7834 (n=3; biological replicates). Statistical significance was determined by using one- or two-way analysis of variance following a Bonferroni multiple comparison posttest. *P<0.05; **P<0.01; ***P<0.001. BFU-E, burst forming unit-erythroid; CFC, colony forming cell; MTL, mean telomere length; n.s., not significant.

FIG. 3. RG7834 rescues TERC levels and telomerase activity in HeLa cells with depleted DKC1 and PARN. (A) Quantification of DKC1 and PARN by qRT-PCR in HeLa cells under the indicated conditions. (n=3; biological replicates). Values are expressed in relation to Scrambled control. (B) Western-blot analysis of DKC1 and PARN levels in HeLa cells under the indicated conditions. (C) Quantification of TERC levels by qRT-PCR in HeLa cells treated with DMSO or RG7834 at the indicated concentration (n=3; biological replicates). (D) Quantification of TRAP analysis shown in FIG. 1D. Values are expressed in relation to Scrambled control; (n=3).

FIG. 4. Increased TERC levels and telomerase activity in DKC1_A353V hESCs treated with RG7834. (A) Quantification of TERC and TERT by qRT-PCR in DKC1_A353V hESCs treated with DMSO or different concentrations of RG7834 for 4 days (n=3; biological replicates). (B) Quantification of TRAP analysis shown in FIG. 2C. Values are expressed in relation to WT hESCs; (n=3).

FIG. 5. No detectable RG7834 toxicity in hESCs. (A) Representative immunoblot analysis of γH2AX in DKC1_A353V hESCs treated with different concentrations of RG7834 (indicated in the figure). Positive control: Etoposide 50 nM. Actin is shown as a loading control. (B) Quantification of NANOG by qRT-PCR in DKC1_A353V hESCs treated with DMSO or different concentrations of RG7834 (as indicated) for 60 days. (C) Flow cytometry analysis of EdU incorporation with DNA content (DAPI) in DKC1_A353V hESCs, treated with DMSO or 5 μM of RG7834 for 60 days. Quantification of active proliferating cells (EdU positive) is indicated. (D) MA plot for the analysis of RNA-sequencing experiments between DMSO or RG7834 treated DKC1_A353V hESCs. No significant differences in gene expression were found.

FIG. 6. In vitro definitive hematopoietic differentiation schematic. hESCs are plated on Day 0 in MEF (mouse embryonic fibroblasts) coated plates. WNT activation by CHIR99021 and SB-431542 treatment at Day 2 of differentiation determines definitive hematopoietic specification. Definitive mesoderm specification is assessed at Day 3 by KDR and CD235a expression. CD34+CD43− cells are assayed at Day 8. Hematopoietic colony potential is assessed at Day 28.

FIG. 7. Chemical structure of RG7834.

FIG. 8. Schematic diagram showing the chemical inhibition of PAPD5/7 rescues telomerase function and hematopoiesis in dyskeratosis congenita.

### DETAILED DESCRIPTION OF INVENTION

This disclosure provides novel therapeutic strategies for the treatment of a telomere-associated disease or disorder through the inhibition of poly(A) polymerase PAPD5. In one preferred embodiment, the invention provides novel therapeutic strategies for the treatment of a telomere-associated disease or disorder using the small molecule RG7834 (FIG. 7) to inhibit the poly(A) polymerase PAPD5.

As used herein and in the appended claims, the singular forms “a”, “and”, and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth.

The term “telomere associated gene” is meant to refer to a gene that is part of the telomere complex or a gene that encodes any member of the telomere pathway.

The term “gene” is meant to refer to a segment of nucleic acid that contains the information necessary to produce a functional RNA product. A gene usually contains regulatory regions dictating under what conditions the RNA product is made, transcribed regions dictating the sequence of the RNA product, and/or other functional sequence regions. A gene may be transcribed to produce an mRNA molecule, which contains the information necessary for translation into the amino acid sequence of the resulting protein.

The phrase “telomere associate disease or disorder” is meant to refer to any disease, condition, or disorder that is caused by an alteration in a gene associated with the telomere or the telomere pathway. A telomere associated disease or disorder can also refer to any disease, condition or disorder that is caused by shortening of the telomere. In certain preferred examples, a telomere associated disease or disorder can be dyskeratosis congenita, aplastic anemia and myelodysplastic syndrome, or hematopoietic defects.

The term “dyskeratosis congenita” is meant to refer to a hereditary disorder with features that include, but are not limited to, cutaneous pigmentation, dystrophy of the nails, leukoplakia of the oral mucosa and low blood counts.

The term “compound” or “composition” “a compound of the invention” includes all solvates, complexes, polymorphs, radiolabeled derivatives, tautomers, stereoisomers, and optical isomers of compounds that inhibit PAPD5. For example, the invention includes all solvates, complexes, polymorphs, radiolabeled derivatives, tautomers, stereoisomers, and optical isomers of the PAPD5 inhibitor compound RG7834 generally described herein, and salts thereof, unless otherwise specified.

The term “telomerase RNA” or TERC is meant to refer to the telomerase RNA component. In certain cases, TERC is known as hTR. In certain embodiments, the sequence of full-length native TERC is represented by GenBank Accession No. U85256/NR-001566. For example, the sequence of Homo sapiens telomerase (TERC) is shown below and comprises SEQ ID NO: 2.

GGGTTGCGGAGGGTGGGCCTGGGAGGGGTGGTGGCC
ATTTTTTGTCTAACCCTAACTGAGAAGGGCGTAGG
CGCCGTGCTTTTGCTCCCCGCGCGCTGTTTTTCTC
GCTGACTTTCAGCGGGCGGAAAAGCCTCGGCCTGC
CGCCTTCCACCGTTCATTCTAGAGCAAACAAAAAA
TGTCAGCTGCTGGCCCGTTCGCCCCTCCCGGGGAC
CTGCGGCGGGTCGCCTGCCCAGCCCCCGAACCCCG
CCTGGAGGCCGCGGTCGGCCCGGGGCTTCTCCGGA
GGCACCCACTGCCACCGCGAAGAGTTGGGCTCTGT
CAGCCGCGGGTCTCTCGGGGGCGAGGGCGAGGTTC
AGGCCTTTCAGGCCGCAGGAAGAGGAACGGAGCGA
GTCCCCGCGCGCGGCGCGATTCCCTGAGCTGTGGG
ACGTGCACCCAGGACTCGGCTCACACATGC

The terms “individual,” “subject,” and “patient,” used interchangeably herein, refer to a mammal, including, but not limited to, murines, simians, humans, mammalian farm animals, mammalian sport animals, and mammalian pets. Preferably, the subject herein is human.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, and particularly in a human, and includes: (a) preventing the disease from occurring in a subject which may be predisposed to the disease but has not yet been diagnosed as having it: (b) inhibiting the disease, i.e., arresting its development; (c) relieving the disease, i.e., causing regression of the disease; (d) protection from or relief of a symptom or pathology caused by TERC activity; (e) reduction, decrease, inhibition, amelioration, or prevention of onset, severity, duration, progression, frequency or probability of one or more symptoms or pathologies associated with TERC activity; and (f) prevention or inhibition of a worsening or progression of symptoms or pathologies associated with TERC activity.

Reduction in telomerase RNA component (TERC) levels due to genetic mutations in DKC1 (dyskerin), TERC (telomerase RNA) or PARN (Poly(A) ribonuclease) lead to a family of bone marrow failure diseases such as Dyskeratosis Congenita (DC), Aplastic Anemia (AA) and Myelodysplastic syndrome (MDS). Importantly, some of these mutations do not affect the function of TERC in cells but only its stability. The present inventors have previously shown that stabilization of TERC levels by reducing its 3′ end modification by the polymerase PAPD5 can rescue TERC function in cancer and stem cells, and lead to telomere elongation and phenotypic rescue in hematopoietic stem cells. This suggests that PAPD5 inhibition is a potent therapeutic strategy for treatment of DC and other bone marrow failure diseases. Currently, there are no known small molecule inhibitors of PAPD5 and the only available treatment for DC consists of expensive bone marrow transplants with poor success rate. This highlights an urgent need for new therapies that can benefit DC patients.

In one embodiment of the current invention, the present inventors may inhibit the activity of PAPD5 in a subject in need thereof, for example through a small-molecule inhibitor compound. In this embodiment, a therapeutically effective amount of a PAPD5 inhibitor may be administered to a subject in need thereof. In this embodiment, the therapeutically effective amount of a PAPD5 inhibitor may inhibit the activity of PAPD5 in said subject. Inhibition of PAPD5 may produce one or more of the following therapeutic actions:
rescue human telomerase RNA component (TERC) function in the subject;
inhibit degradation of TERC in the subject;
increase levels of TERC in the subject;
increase telomerase activity in the subject;
increase telomere homeostasis in the subject;
modulate localization of TERC in the subject;
inhibit DNA damage signaling arising from eroded telomeres in the subject;
increase localization of TERC to the cajal bodies of the nucleus of one or more cells of the subject;
increase telomere length, and preferably increase telomere lengths in human embryonic stem cells (hESCs) carrying the DKC1 A353V mutation;
increase hematopoietic differentiation, and preferably hematopoietic potential in hESCs carrying the DKC1_A353V mutants;
increase telomere lengths in DKC1 depleted cells of the subject;
inhibit 3′ end oligoadenylation of TERC in the subject; and
increase telomere lengths in PARN depleted cells of the subject.

In a preferred embodiment of the current invention, the present inventors use the small molecule compound RG7834, which was originally discovered as a Hepatitis B therapy and later identified as a PAPD5/PAPD7 inhibitor, to rescues TERC levels in DC cell culture models. RG7834 was found to be highly active in cell culture against the HPV B infection and exhibited low toxicity in cell culture models, suggesting a specific mode of action in cells.

As such, in another embodiment of the current invention, the present inventors may inhibit the activity of PAPD5 in a subject in need thereof, for example through the action of the PAPD5 inhibitor RG7834. In this embodiment, a therapeutically effective amount of RG7834 may be administered to a subject in need thereof. In this embodiment, the therapeutically effective amount of RG7834 may inhibit the activity of PAPD5 in said subject. Inhibition of PAPD5 by therapeutically effective amount of RG7834 may produce one or more of the following therapeutic actions:
rescue human telomerase RNA component (TERC) function in the subject;
inhibit degradation of TERC in the subject;
increase levels of TERC in the subject;
increase telomerase activity in the subject;
increase telomere homeostasis in the subject;
modulate localization of TERC in the subject;
inhibit DNA damage signaling arising from eroded telomeres in the subject;
increase localization of TERC to the cajal bodies of the nucleus of one or more cells of the subject;
increase telomere length, and preferably increase telomere lengths in human embryonic stem cells (hESCs) carrying the DKC1 A353V mutation;
increase hematopoietic differentiation, and preferably hematopoietic potential in hESCs carrying the DKC1_A353V mutants;
increase telomere lengths in DKC1 depleted cells of the subject;
inhibit 3′ end oligoadenylation of TERC in the subject; and
increase telomere lengths in PARN depleted cells of the subject.

In one embodiment, the levels of DKC1 or PARN in HeLa cells were reduced using siRNAs and then treated them with DMSO or RG7834 for two days. The present inventors found that TERC levels were reduced in DKC1 and PARN knockdown cells, as expected. Importantly, treatment with RG7834 for two days completely rescued TERC levels in PARN depleted cells compared to DMSO treatment, and partially rescued TERC levels in DKC1 depleted cells. No cellular toxicity was observed in cell culture models, suggesting that RG7834 is well tolerated. In another embodiment, the present inventors demonstrate that RG7834 rescues TERC levels in a physiological model of DC. This model included previously characterized stem cells carrying disease-causing mutations in the DKC1 gene. The present inventors found that RG7834 treatment partially rescued TERC levels in the stem cell model of DC, similar to what was observed in HeLa cells. Based on previous work by the inventors, these data suggest that RG7834 treatment can reverse the molecular hallmark of some DC patients, i.e., reduction in TERC levels, and potentially rescues subsequent decrease in telomerase activity and loss of telomeres at chromosome ends.

Thus, this disclosure provides methods of reducing the severity of one or more symptom(s) of a telomere-associated disease or disorder in a subject in need thereof and/or identifying a subject that may selectively benefit from the administration of one or more PAPD5 inhibitor(s). In one preferred embodiment, a PAPD5 inhibitor may include a therapeutically effective amount of RG7834.

For example, in one preferred embodiment of the current invention, the present inventors may treat a telomere-associate disease or condition in a subject in need thereof, through the inhibition of PAPD5 for example through a small-molecule inhibitor compound. In this embodiment, a therapeutically effective amount of a PAPD5 inhibitor may be administered to a subject suffering from, or at risk of developing a telomere-associate disease or condition. In this embodiment, the therapeutically effective amount of a PAPD5 inhibitor may inhibit the activity of PAPD5 in said subject. Inhibition of PAPD5 may inhibit, arrest of ameliorate one or more of the following indications of a telomere-associate disease or condition:
loss of normal human telomerase RNA component (TERC) function in the subject;
atypical degradation of TERC in the subject;
decreased levels of TERC in the subject;
decreased telomerase activity in the subject;
decreased telomere homeostasis in the subject;
atypical localization of TERC in the subject;
increased DNA damage signaling arising from eroded telomeres in the subject;
decreased hematopoietic differentiation, and preferably hematopoietic potential in hESCs carrying the DKC1_A353V mutants;
decreased localization of TERC to the cajal bodies of the nucleus of one or more cells of the subject;
decreased telomere lengths, and in particular decreased telomere lengths in human embryonic stem cells (hESCs) carrying the DKC1 A353V mutation;
increased 3′ end oligoadenylation of TERC in the subject;
decreased telomere lengths in DKC1 depleted cells of the subject; and
decreased telomere lengths in PARN depleted cells of the subject.

In another preferred embodiment of the current invention, the present inventors may treat a telomere-associate disease or condition in a subject in need thereof, through the inhibition of PAPD5 for example through administration of therapeutically effective amount of RG7834. In this embodiment, a therapeutically effective amount of RG7834 may be administered to a subject suffering from, or at risk of developing a telomere-associate disease or condition. In this embodiment, the therapeutically effective amount of RG7834 may inhibit the activity of PAPD5 in said subject. Inhibition of PAPD5 by RG7834 may inhibit, arrest of ameliorate one or more of the following indications of a telomere-associate disease or condition:
loss of normal human telomerase RNA component (TERC) function in the subject;
atypical degradation of TERC in the subject;
decreased levels of TERC in the subject;
decreased telomerase activity in the subject;
decreased telomere homeostasis in the subject;
atypical localization of TERC in the subject;
increased DNA damage signaling arising from eroded telomeres in the subject;
decreased hematopoietic differentiation, and preferably hematopoietic potential in hESCs carrying the DKC1_A353V mutants;
decreased localization of TERC to the cajal bodies of the nucleus of one or more cells of the subject;
decreased telomere lengths, and in particular decreased telomere lengths in human embryonic stem cells (hESCs) carrying the DKC1 A353V mutation;
increased 3′ end oligoadenylation of TERC in the subject;
decreased telomere lengths in DKC1 depleted cells of the subject; and
decreased telomere lengths in PARN depleted cells of the subject.

In one embodiment, PAPD5 inhibition may be indicated by decreased activity (e.g., by at least 10%, 20%, 30%, 40%, 50%, 60%, 70%, 80%, or 90+%).

In another embodiment, the inventive technology includes methods and compositions for the treatment of one or more telomere-associated disease or disorder in a subject in need thereof, which may preferably include the administration of therapeutically effective amount of one or more compositions that may cause the reduction in TERC levels, and may further rescue subsequent decrease in telomerase activity and loss of telomeres at chromosome ends. In one preferred embodiment, a composition that may cause the reduction in TERC levels may include a therapeutically effective amount of RG7834.

In another embodiment, the inventive technology includes methods and compositions for the treatment of telomere-associated disease or disorder in a subject in need thereof, such as DC, aplastic anemia, myelodysplastic syndrome, or familial pulmonary fibrosis, which may be caused by reduced or disrupted TERC processing. Some examples of mutations in telomere maintenance genes that lead to these diseases include mutations in telomere associated genes, such as DKC1, TERC, TERT, TCABJ, PARN, and ZCCHC8. In this preferred embodiment, the invention may include the administration of therapeutically effective amount of one or more compositions that may inhibit TERC degradation and rescue TERC function. In one preferred embodiment, a composition that may inhibit TERC degradation and rescue TERC function may include a therapeutically effective amount of RG7834.

In another embodiment, the inventive technology includes one or more Pharmaceutical compositions that may include a therapeutically effective amount of RG7834. “Pharmaceutical compositions” are compositions that include an amount (for example, a unit dosage) of the disclosed compound(s) together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

In another embodiment, a compound of the invention, and preferably RG7834 may be in the form of a pharmaceutically acceptable salt or ester. The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid, and the like.

A “therapeutically effective amount” of the disclosed compound, which may preferably be RG7834, is a dosage of the compound that is sufficient to achieve a desired therapeutic effect, such as inhibition of or PAPD5 and/or PAPD5/PAPD7, inhibit TERC degradation and/or rescue TERC function, or treatment of a telomere-associated disease or disorder. In some examples, a therapeutically effective amount is an amount sufficient to achieve tissue concentrations at the site of action that are similar to those that are shown to modulate TERC degradation, or PAPD5/PAPD7 activity in tissue culture, in vitro, or in vivo. For example, a therapeutically effective amount of a compound, and preferably RG7834, may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day.

Such pharmaceutical compositions/formulations are useful for administration to a subject, in vivo or ex vivo. Pharmaceutical compositions and formulations include carriers or excipients for administration to a subject. As used herein the terms “pharmaceutically acceptable” and “physiologically acceptable” mean a biologically compatible formulation, gaseous, liquid, or solid, or mixture thereof, which is suitable for one or more routes of administration, in vivo delivery, or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules, and crystals. Supplementary active compounds (e.g., preservatives, antibacterial, antiviral, and antifungal agents) can also be incorporated into the compositions. The formulations may, for convenience, be prepared or provided as a unit dosage form. In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone. Supplementary active compounds (e.g., preservatives, antioxidants, antimicrobial agents including biocides and biostats such as antibacterial, antiviral, and antifungal agents) can also be incorporated into the compositions. Preservatives and other additives include, for example, antimicrobials, anti-oxidants, chelating agents, and inert gases (e.g., nitrogen). Pharmaceutical compositions may therefore include preservatives, antimicrobial agents, anti-oxidants, chelating agents, and inert gases.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical compositions can optionally be formulated to be compatible with a particular route of administration. Exemplary routes of administration include administration to a biological fluid, an immune cell (e.g., T or B cell) or tissue, mucosal cell or tissue (e.g., mouth, buccal cavity, labia, nasopharynx, esophagus, trachea, lung, stomach, small intestine, vagina, rectum, or colon), neural cell or tissue (e.g., ganglia, motor or sensory neurons) or epithelial cell or tissue (e.g., nose, fingers, ears, cornea, conjunctiva, skin or dermis). Thus, pharmaceutical compositions include carriers (excipients, diluents, vehicles, or filling agents) suitable for administration to any cell, tissue, or organ, in vivo, ex vivo (e.g., tissue or organ transplant) or in vitro, by various routes and delivery, locally, regionally, or systemically.

Exemplary routes of administration for contact or in vivo delivery of a PAPD5/PAPD7 inhibitor, such as RG7834, is a dosage of the compound that is sufficient to achieve a desired therapeutic effect, such as can optionally be formulated include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, and intralymphatic.

Formulations suitable for parenteral administration include aqueous and non-aqueous solutions, suspensions, or emulsions of the compound, which may include suspending agents and thickening agents, which preparations are typically sterile and can be isotonic with the blood of the intended recipient. Non-limiting illustrative examples of aqueous carriers include water, saline (sodium chloride solution), dextrose (e.g., Ringer's dextrose), lactated Ringer's, fructose, ethanol, animal, vegetable, or synthetic oils. Examples of non-aqueous solvents are propylene glycol, polyethylene glycol, vegetable oils such as olive oil, and injectable organic esters such as ethyl oleate. Intravenous vehicles include fluid and nutrient replenishers, electrolyte replenishers (such as those based on Ringer's dextrose). The formulations may be presented in unit-dose or multi-dose kits, for example, ampules and vials, and may be stored in a freeze-dried (lyophilized) condition requiring addition of a sterile liquid carrier, for example, water for injections, prior to use.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, pastes, lotions, oils, or creams as generally known in the art.

For topical administration, for example, to skin, pharmaceutical compositions typically include ointments, creams, lotions, pastes, gels, sprays, aerosols, or oils. Carriers which may be used include Vaseline, lanolin, polyethylene glycols, alcohols, transdermal enhancers, and combinations thereof. An exemplary topical delivery system is a transdermal patch containing an active ingredient. For oral administration, pharmaceutical compositions include capsules, cachets, lozenges, tablets, or troches, as powder or granules. Oral administration formulations also include a solution or a suspension (e.g., aqueous liquid or a non-aqueous liquid; or as an oil-in-water liquid emulsion or a water-in-oil emulsion). For airway or nasal administration, pharmaceutical compositions can be formulated in a dry powder for delivery, such as a fine or a coarse powder having a particle size, for example, in the range of 20 to 500 microns which is administered in the manner by inhalation through the airways or nasal passage. Depending on delivery device efficiency, effective dry powder dosage levels typically fall in the range of about 10 to about 100 mg. Appropriate formulations, wherein the carrier is a liquid, for administration, as for example, a nasal spray or as nasal drops, include aqueous or oily solutions of the active ingredient.

For airway or nasal administration, aerosol and spray delivery systems and devices, also referred to as “aerosol generators” and “spray generators,” such as metered dose inhalers (MDI), nebulizers (ultrasonic, electronic, and other nebulizers), nasal sprayers and dry powder inhalers can be used. MDIs typically include an actuator, a metering valve, and a container that holds a suspension or solution, propellant, and surfactant (e.g., oleic acid, sorbitan trioleate, lecithin). Activation of the actuator causes a predetermined amount to be dispensed from the container in the form of an aerosol, which is inhaled by the subject. MDIs typically use liquid propellant and typically, MDIs create droplets that are 15 to 30 microns in diameter, optimized to deliver doses of 1 microgram to 10 mg of a therapeutic. Nebulizers are devices that turn medication into a fine mist inhalable by a subject through a face mask that covers the mouth and nose. Nebulizers provide small droplets and high mass output for delivery to upper and lower respiratory airways. Typically, nebulizers create droplets down to about 1 micron in diameter.

Dry-powder inhalers (DPI) can be used to deliver the compounds of the invention, either alone or in combination with a pharmaceutically acceptable carrier. DPIs deliver active ingredient to airways and lungs while the subject inhales through the device. DPIs typically do not contain propellants or other ingredients, only medication, but may optionally include other components. DPIs are typically breath-activated but may involve air or gas pressure to assist delivery.

Pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

In the methods of the invention, a PAPD5/PAPD7 inhibitor, such as RG7834, may be administered in accordance with the methods at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly, or monthly. Timing of contact, administration ex vivo or in vivo delivery can be dictated by the, pathogenesis, symptom, pathology, or adverse side effect to be treated. For example, an amount can be administered to the subject substantially contemporaneously with, or within about 1-60 minutes or hours of the onset of a symptom or adverse side effect of treatment.

Doses may vary depending upon whether the treatment is therapeutic or prophylactic, the onset, progression, severity, frequency, duration, probability of or susceptibility of the symptom, the type of pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the, pathology or symptom, or any adverse side effects of the treatment or therapy. The skilled artisan will appreciate the factors that may influence the dosage, frequency and timing required to provide an amount sufficient or effective for providing a prophylactic or therapeutic effect or benefit.

Doses can be based upon current existing treatment protocols, empirically determined, determined using animal disease models or optionally in human clinical studies. A subject may be administered in single bolus or in divided/metered doses, which can be adjusted to be more or less according to the various consideration set forth herein and known in the art. Dose amount, frequency or duration may be increased or reduced, as indicated by the status of pathogenesis, associated symptom or pathology, or any adverse side effect(s). For example, once control or a particular endpoint is achieved, for example, reducing, decreasing, inhibiting, ameliorating, or preventing onset, severity, duration, progression, frequency, or probability of one or more symptoms associated with a telomere-associated disease or disorder.

Another embodiment of this disclosure provides pharmaceutical kits containing a pharmaceutical composition of this disclosure, and preferably RG7834, prescribing information for the composition, and a container.

Each publication or patent cited herein is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1: RG7834 Treatment Rescues TERC Levels and Localization in DKC1 or PARN Depleted HeLa Cells

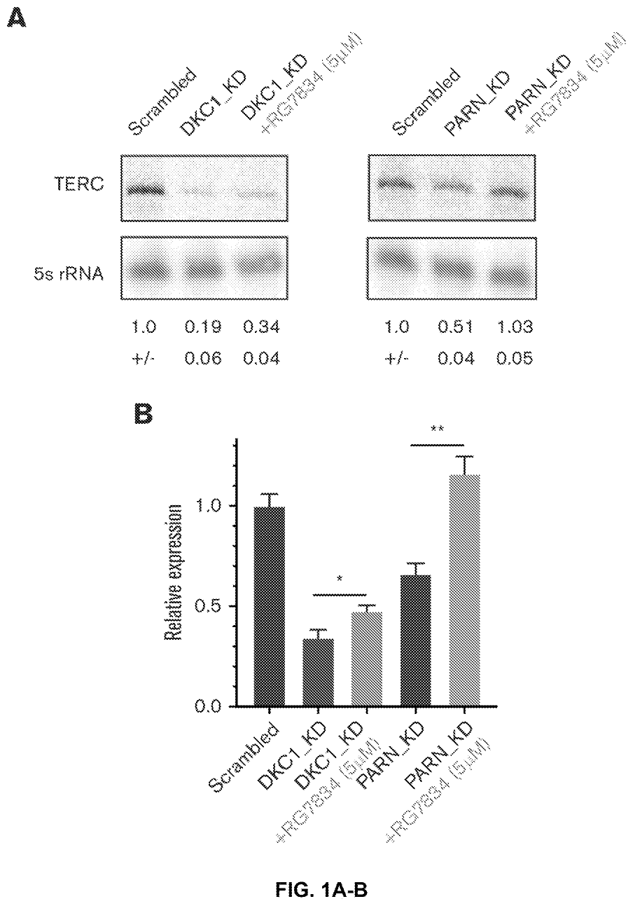

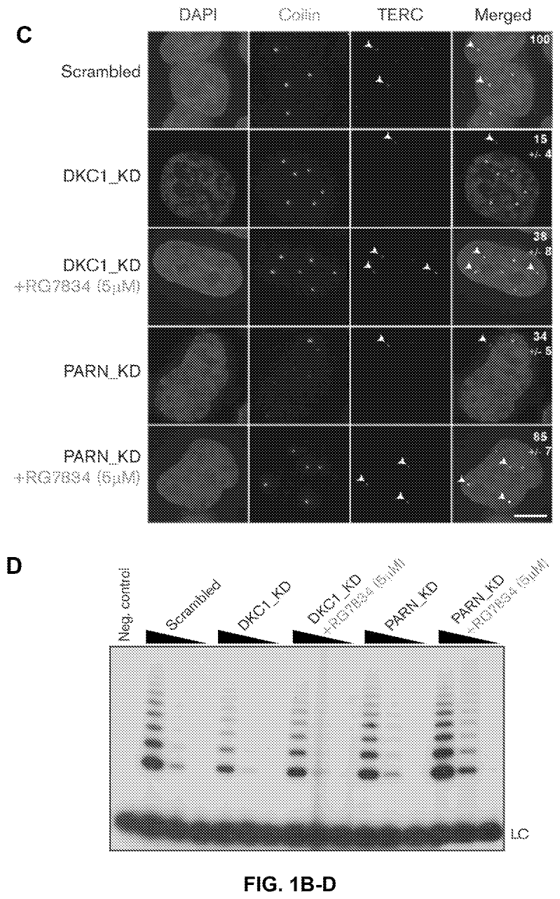

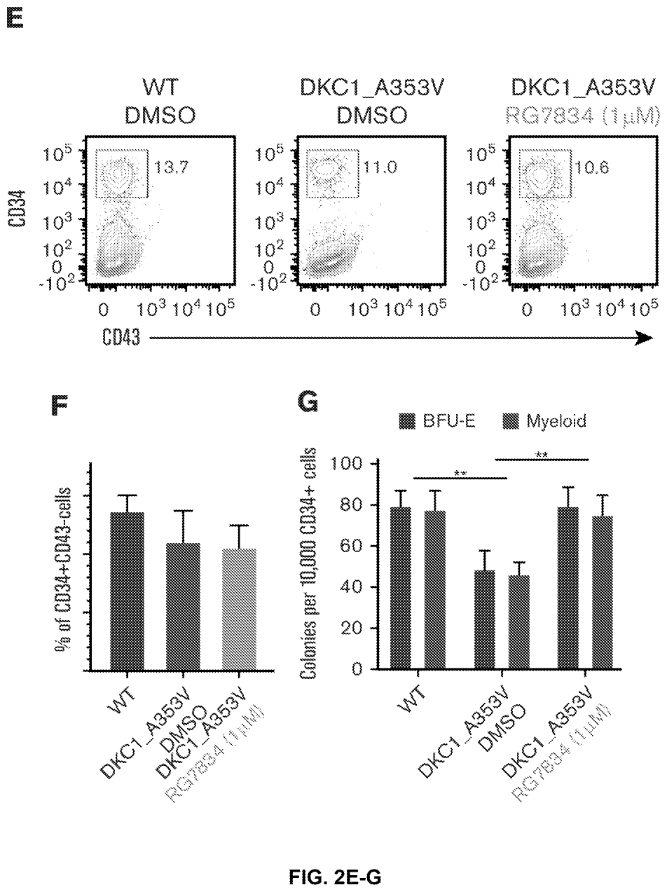

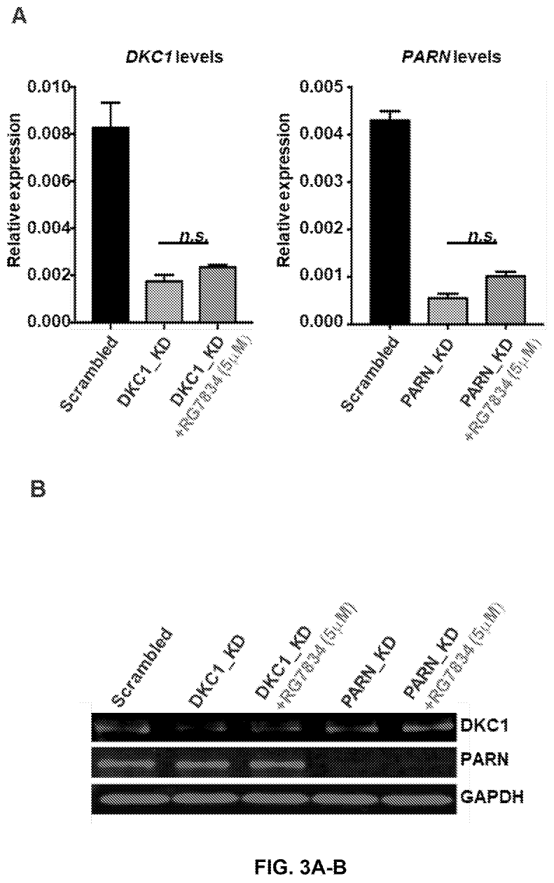

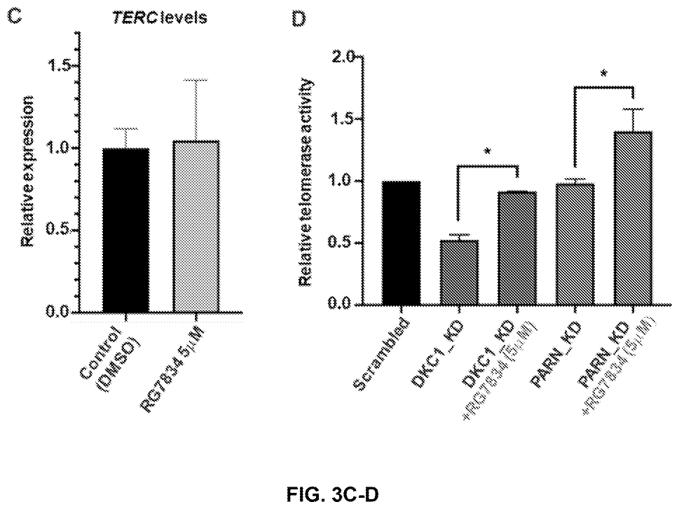

To investigate whether RG7834 treatment phenocopies PAPD5 knockdown, the present inventors depleted DKC1 in HeLa cells (FIG. 3A-B) and treated them with either DMSO (control) or 5 μM of RG7834 for 2 days. As shown in FIGS. 1A-B, knockdown of DKC1 led to a significant reduction in TERC levels. Importantly, treatment with RG7834 led to a significant increase in TERC levels in DKC1 knockdown cells, indicating that RG7834 treatment increased TERC levels in DKC1-deficient cells. Because the depletion of PARN also leads to TERC degradation mediated by 3′ end adenylation by PAPD5, the present inventors silenced PARN in HeLa cells and treated them with DMSO or 5 μM of RG7834 for 2 days. PARN knockdown led to a significant reduction in TERC levels, which is completely rescued by RG7834 treatment. Thus, similar to the genetic silencing of PAPD5, treatment with RG7834 increased TERC levels in both PARN- and DKC1-deficient cells, without affecting levels of either DKC1 or PARN in individual knockdown cells. Finally, RG7834 treatment did not affect TERC levels in HeLa cells that were not subject to silencing of either DKC1 or PARN (FIG. 3C).

Th present inventors have previously shown that in DKC1- or PARN-depleted cells, TERC accumulates in cytoplasmic puncta called “cyTER bodies,” instead of its normal localization to cajal bodies in the nucleus. To investigate whether RG7834 treatment rescued TERC localization to cajal bodies in the nucleus, DKC1- or PARN-depleted cells were treated with RG7834 for 2 days. In WT control cells, TERC localized to cajal bodies, suggesting normal accumulation and trafficking of the telomerase RNA component (FIG. 1C). However, in control DKC1 knockdown cells, only ˜15% of cells showed TERC localization to cajal bodies, with a significant number of cells exhibiting TERC accumulation in cyTER bodies. Upon treatment with RG7834, —38% of cells had TERC localized to cajal bodies, which is consistent with the 1.5× increase in TERC levels in these cells. RG7834 treatment also rescued TERC localization to cajal bodies in PARN-depleted cells. In PARN knockdown cells, ˜34% of cells had TERC in cajal bodies, with TERC also mostly being localized to cyTER bodies in these cells. However, upon RG7834 treatment, ˜85% of cells had TERC localized to cajal bodies. Finally, treatment with RG7834 was also able to increase telomerase activity in DKC1- and PARN-depleted cells (detected by using 2-step telomere repeat amplification assays) (FIG. 1D; FIG. 3D), which is consistent with the increase in TERC levels (FIG. 1A-B). No toxicity was observed in RG7834-treated cells for the duration of these experiments. Taken together, these results suggest that the chemical inhibition of RG7834 is effective at increasing TERC levels, correcting TERC localization to cajal bodies, and increasing telomerase activity in DKC1- or PARN-depleted cells, indicating restored telomerase function in these conditions.

### Example 2: RG7834 Treatment Rescues TERC Levels and Increases Telomere Length in DKC1 A353V Mutant hESCs

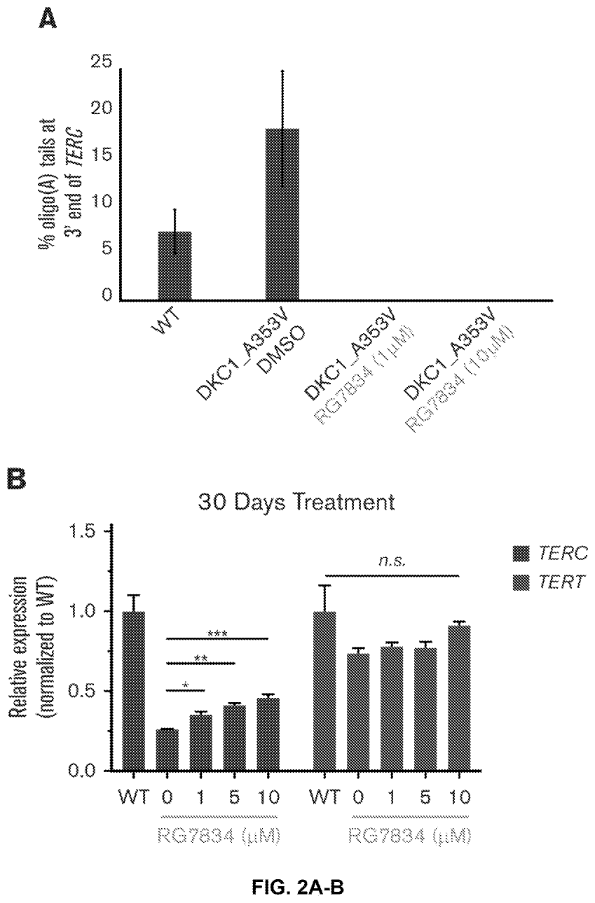

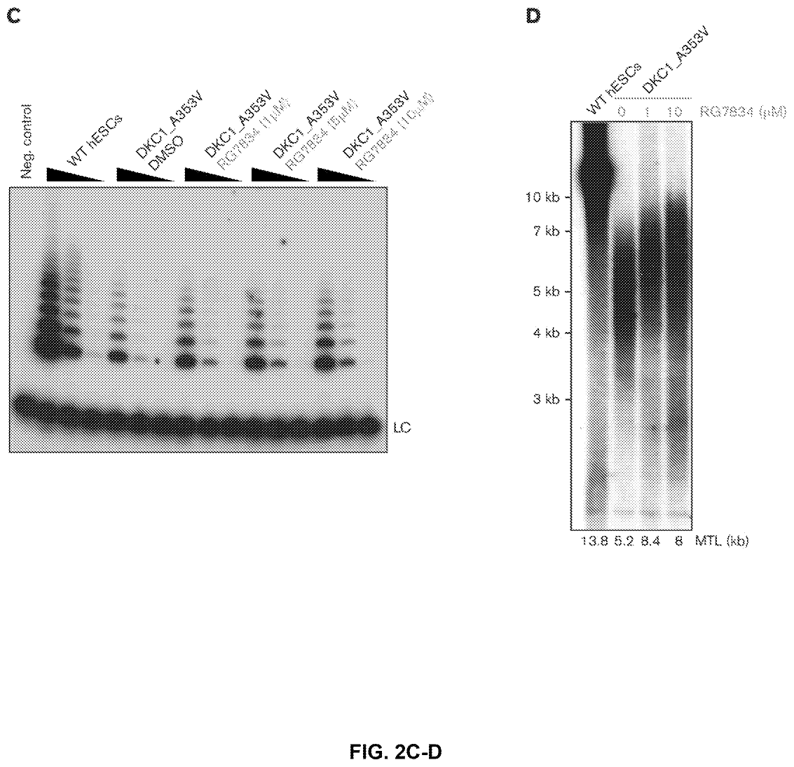

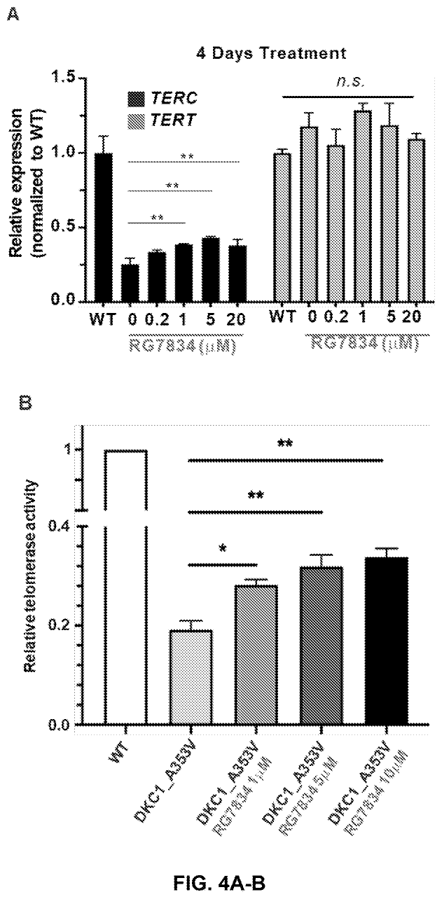

The present inventors next investigated whether RG7834 exhibits a similar effect on TERC levels and function in a physiologically relevant model of DC. A model was previously generated (through CRISPR/Cas9 gene editing) having a DKC1_A353V mutant hESCs, which exhibit reduced TERC levels and telomerase activity, progressive telomere shortening, and impaired definitive hematopoietic specification. Treatment of DKC1_A353V hESCs with RG7834 caused a >15-fold reduction in the 3′-end oligoadenylation of TERC, indicating efficient PAPD5 inhibition in these cells (FIG. 2A). Inhibition of 3′ end oligoadenylation led to a significant increase in TERC levels in DKC1_A353V hESCs (4 days of treatment) (FIG. 4A), which was sustained up to 30 days upon continuous treatment with different concentrations of RG7834 (FIG. 2B). Although the levels of the telomerase reverse transcriptase (TERT) remained unchanged during this period, the increased TERC levels found in DKC1_A353V hESCs treated with RG7834 caused a noticeable rise in telomerase activity in DKC1_A353V hESCS treated with different concentrations of the PAPD5/7 inhibitor (FIG. 2C; FIG. 4B).

The sustained treatment (up to 3 months) with RG7834 also led to improved telomere maintenance, analyzed by telomere restriction fragment analysis, in DKC1_A353V hESCs (FIG. 2D). Together, these data suggest that RG7834 treatment rescued TERC levels, prevented its 3′ end oligoadenylation, increased telomerase activity, and improved telomere homeostasis in DKC1_A353V hESCs.

### Example 3: RG7834 Treatment Reduced DNA Damage Signaling Arising from Eroded Telomeres

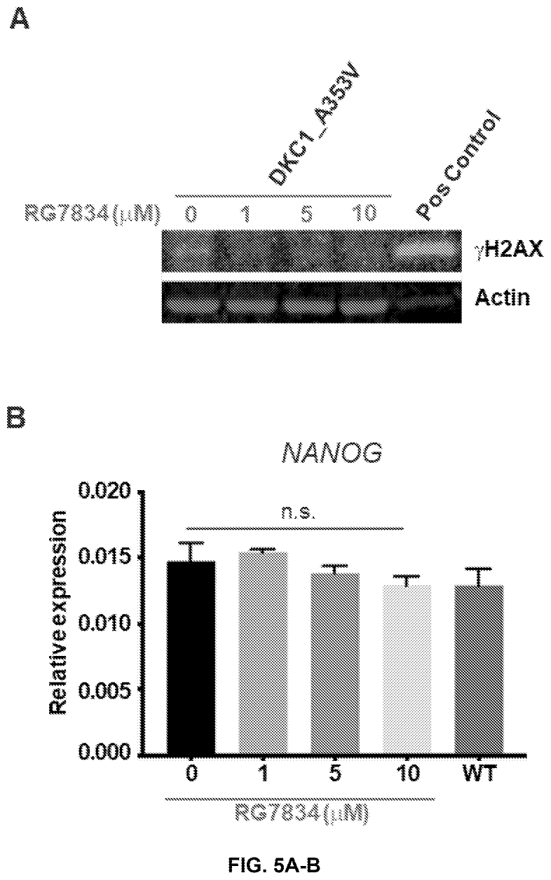

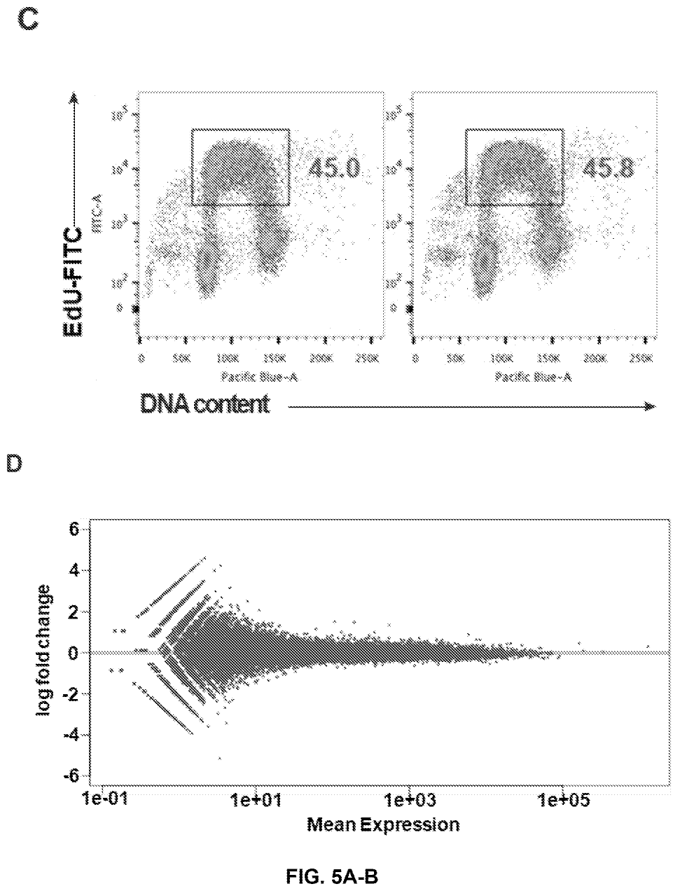

The present inventors investigated if treatment with RG7834 was sufficient to reduce DNA damage signaling arising from eroded telomeres, a hallmark of DC. It was observed that γH2AX levels were reduced in DKC1_A353V cells treated with different concentrations of RG7834 compared with DMSO-treated cells (FIG. 5A). Importantly, no toxicity associated with RG7834 treatment was detected at the concentrations indicated, during the entire duration of the experiments performed (FIG. 5B-C). In addition, whole-genome RNA-sequencing analysis showed that treatment with 1 μM of RG7834 did not lead to significant changes in gene expression in DKC1_A353V mutant hESCs compared with DMSO-treated cells (FIG. 5D), suggesting that RG7834 treatment affects specific RNAs in hESCs and rules out toxicity associated with genome-wide gene expression perturbations. Combined, these data indicate that similar to the genetic silencing of PAPD5, treatment with RG7834 is able to rescue the major biochemical phenotypes observed in DC models.

### Example 4: RG7834 Treatment Enhances Hematopoietic Potential in DKC1_A353V Mutants

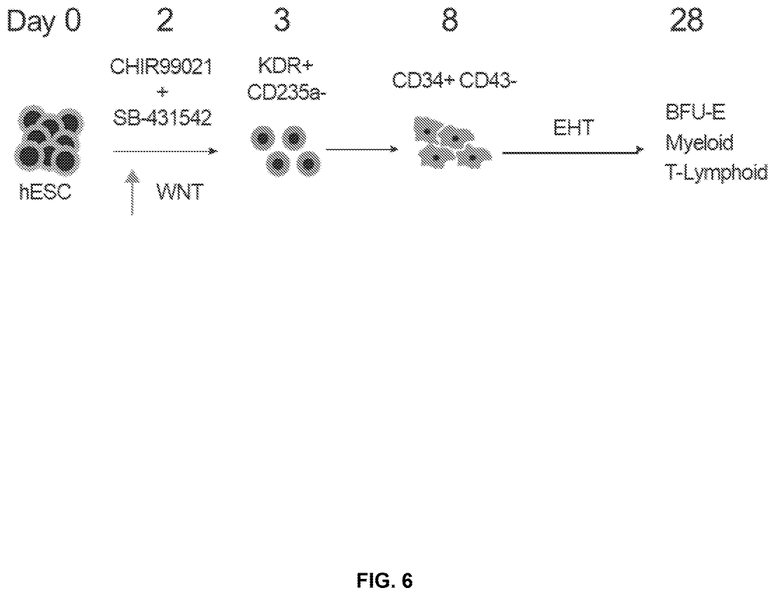

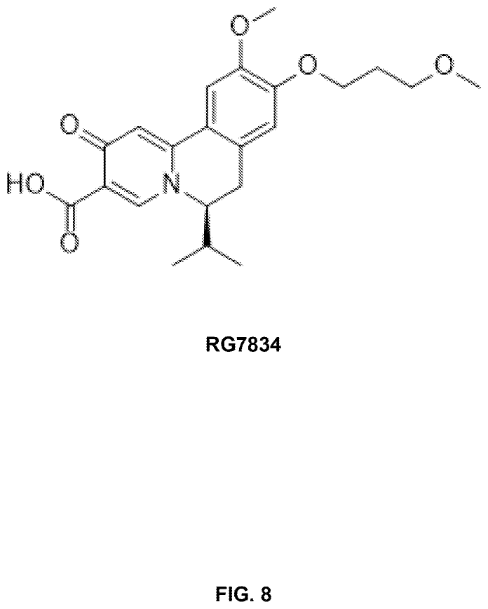

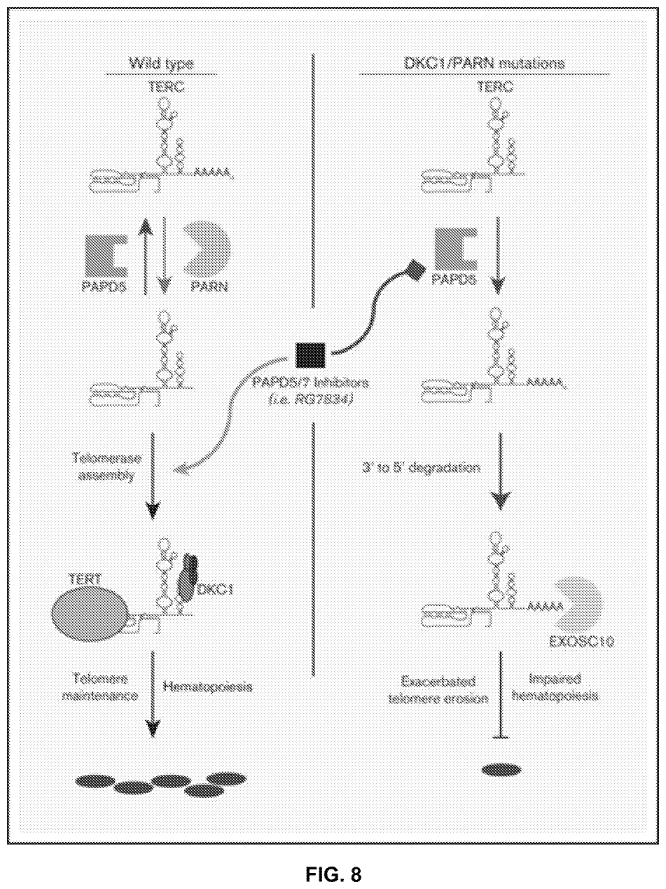

Because bone marrow failure is the leading cause of death in DC, the present inventors wanted to analyze the consequences of RG7834 treatment in DKC1_A353V cells during definitive hematopoietic differentiation. There are no animal models that faithfully recapitulate the hematopoietic defects observed in patients harboring pathogenic mutations in DKC1 or PARN; therefore, the present inventors followed established protocols of hESC differentiation into hematopoietic lineages. These protocols recapitulate, in vitro, the major aspects of blood development in vivo, a strategy shown to accurately model key aspects of DC. A schematic of the protocol is depicted in FIG. 6. These data show that although CD34⁺CD43⁻ early hematopoietic progenitors (day 8 of differentiation) (FIG. 2E-F) were similar in all samples, definitive hematopoietic colony potential analysis (day 28 of differentiation) revealed that treatment with different concentrations of RG7834 significantly increased the hematopoietic potential of DKC1_A353V cells (FIG. 2G). These observations provide compelling evidence that chemical inhibition of PAPD5, in addition to rescuing telomerase function, is sufficient to increase definitive, multilineage, hematopoietic potential in DKC1_A353V mutants.

### Example 5: Inhibition of PAPD5/7 Significantly Increases TERC Levels, Localization, and Function in DKC1- and PARN-Deficient Cells

The present inventors show that RG7834 reduces the 3′-end oligoadenylation of TERC, increases TERC levels and telomerase activity, and elongates telomeres in PARN- or DKC1-deficient cells. RG7834 treatment was sufficient to restore the in vitro definitive hematopoietic development of DKC1_A353V hESCs, similarly to what has recently observed with the genetic silencing of PAPD5. Although these experiments represent the first nongenetic rescue of hematopoietic development from hESCs in DKC1 mutant cells, it has recently been shown that PAPD5 inhibitors are able to promote telomere restoration in different patient-derived DC samples in vitro and in vivo, through xenotransplantation assays.

In addition, the inventive technology presented here indicate that small increases in TERC levels (less than twofold) are sufficient to increase telomerase activity and improve hematopoietic output in DKC1 mutant cells. Additional embodiments of the invention may include the treatment for DC in cells/subjects harboring mutations in other genes that impair TERC levels/function (including TERC itself, NHP2, NOP10, and ZCCHC8) with a PAPD5 inhibitor, such as RG7834 treatment. In addition, the efficiency of PAPD5 inhibition to ameliorate other phenotypes that are commonly associated with telomere shortening, such as pulmonary fibrosis and liver disease, must be assessed, as these conditions cause substantial morbidity and mortality and represent an important unmet need for these patients. In sum, the experiments presented here indicate that the chemical inhibition of PAPD5 by RG7834 or other specific small molecule inhibitors can be a promising therapeutic approach for the treatment of DC or other telomere biology syndromes caused by mutations that reduce TERC levels.

### Example 6: Materials and Methods

Cell culture: hESCs (H1; WA01) were obtained from WiCell Research Institute (Madison, Wis.), following all institutional guidelines determined by the Embryonic Stem Cell Research Oversight Committee (ESCRO) at Washington University in St. Louis. hESCs were maintained in mTeSR1 medium (StemCell Technologies, Vancouver, Canada) on plates coated with Matrigel (BD Biosciences, San Jose, Calif.) diluted at 1:80 in hESC basal medium (DMEM/F12 supplemented with 1% non-essential amino acids, 0.1% beta-mercaptoethanol, and 0.1% pen/strep, Invitrogen). For feeder-based conditions, hESC basal medium with 20% KnockOut Serum Replacement and 10 ng/ml bFGF was used as a medium on growth-arrested mouse embryonic fibroblasts (MEFs). hESCs were cultured in a humidified incubator at 37° C. in 5% CO2 and 5% O2 levels. HeLa cells were obtained from ATCC and cultured in Dulbecco's Minimum Essential Medium (DMEM) supplemented with 10% Fetal Bovine Serum (FBS), 1× Glutamax, 1× Pen/Strep and Normocin. HeLa cells were cultured in a humidified incubator at 37° C. in 5% CO2.

Gene editing: DKC1_A353V (X-linked) mutant hESCs were generated using CRISPR/Cas9 genome editing technology as described previously²¹. Briefly, CRISPR gRNAs were inserted into the MLM3636 plasmid (Addgene #43860) and transfected with Cas9 plasmid (Addgene #43945) and single-stranded DNA donor oligos using Lonza's 4D-Nucleofector with the P4 Primary Cell 4D-Necleofector kit (Allendale, N.J.). Cells were seeded at low density, picked manually, and then sequenced.

siRNA transfection and Northern blotting: ˜120,000 cells were seeded per well in a 6-well plate 24 hours before transfection. Cells were transfected with 5 μM final concentration (0.5 μM for DKC1 siRNA) of siRNAs using Interferin (Polyplus) as per manufacturer's protocol. siRNAs used in this study were purchased from following suppliers: All-stars negative control siRNA (Qiagen), HS_DKC1_1 (Qiagen), siGenome Human PARN (Horizon Discovery). Cells were treated with RG7834 or equivalent volume of DMSO 24 hours post-transfection. Cells were harvested three days after transfection using Trizol (Thermo Fisher). RNA was extracted as per manufacturer's protocol and quantified on a NanoDrop. 10 μg of total RNA was separated on a 5% polyacrylamide 7M Urea gel at 20 W. The RNA was transferred to a nylon membrane (Nytran SPC) using wet transfer in 0.5× TBE at 30V overnight, and the blot was hybridized with 5′-radiolabeled DNA oligos against TERC or 5s rRNA. The blots were imaged on a Typhoon phosphoimager and bands were quantified on Fiji. Intensity of TERC bands were normalized to 5s rRNA.

Fluorescent microscopy for Cajal bodies and TERC: Cells were seeded directly on glass coverslips (ethanol washed) in 6-well plates and transfected and drug treated as previously described. Coverslips were washed once with 1× DPBS and fixed with 4% formaldehyde in 1× DPBS at room temperature for 10 minutes. Cells were permeabilized in 0.2% Triton 1× DPBS for 10 minutes, and then incubated with rabbit anti-Coilin (Santa Cruz) for 1 hour at room temperature. Coverslips were washed thrice with 0.2% Triton 1× DBPS and then incubated with Anti-rabbit Alexa Fluor 488 for 45 minutes. Coverslips were washed again and then fixed with 4% formaldehyde. FISH for TERC was performed as previously described using three 5′-Alexa Fluor 647 labeled TERC probes. Briefly, coverslips were washed twice with 1× PBS and sequentially dehydrated with a 5 minute incubation in 70% ethanol, 95% ethanol and 100% ethanol each. Coverslips were rehydrated in 2× SSC 50% Formamide for 5 minutes and prehybridized at 37 □ C for 30 minutes, followed by hybridization with FISH probes at 37 □ C overnight. Coverslips were washed twice with 2× SSC 50% Formamide for 30 minutes each, followed by a 5 minute wash in 1× PBS. Washed coverslips were cured with Prolong Diamond Antifade Mountant with DAPI. Coverslips were imaged on Deltavision deconvolution microscope and deconvolved images were normalized for the same intensity in each experiment.

3′ end sequencing of TERC: Briefly, ˜100 ng of DNase-treated, rRNA-depleted, Antarctic phosphatase treated RNA was ligated with 40 pmol of barcoded appendix (2 reactions per sample) using T4 RNA Ligase I and 10 mM ATP. Enzymatic reaction was cleaned up using RNA clean and concentrator kit (Zymo). 4 μl of cDNA RNA from each reaction (8 μl total RNA) was used for cDNA synthesis using a primer complementary to the appendix. The cDNA product was then used as template for 5′ RACE using TERC-specific barcoded primers and a universal primer. The RACE product was size-selected on a 1% agarose gel and fragments ˜100-300 bp were gel purified and amplified using Illumina sequencing primers. Libraries were multiplexed and sequenced on an Illumina NextSeq sequencer (mid-output setting and 1×150 cycle kit). Sequenced reads were filtered using both the TERC primer and the appendix barcode and reads with 100% sequence identify were used for downstream analysis. TERC reads with more than 2 As at the mature end (for e.g., UGCAA, UGCAAA, etc.) were quantified as oligoadenylated for analysis.

RNA-seq of transcriptome from DMSO or RG7834 treated DKC1_A353V cells: RNA was extracted from DMSO or RG7834 (1 μM) treated DKC1 mutant cells using Trizol as per manufacturer's instructions and contaminating DNA was removed using TURBO DNase. 1 μg of total RNA in triplicate from each condition was depleted of ribosomal RNA using Ribocop rRNA depletion kit V3 (Lexogen). 50 ng of recovered total RNA was used for strand-specific library preparation using the Rapid Directional RNA-seq kit 2.0 (Perkin Elmer) as per manufacturer's protocol. Prepared libraries were sequenced on an Illumina HiSeq using 2×150 paired end sequencing kit. Demultiplexed reads were checked for quality using the FastQC tool and subjected to adapter trimming using Trimmomatic. Trimmed reads were aligned to the hg38 p13 version 99 human genome release and annotation obtained from ENSEMBL using the STAR alignment software. Aligned files were sorted according to chromosomal coordinate and compressed to BAM files. HTSeq was used to count the number of reads for each gene from BAM files. Read counts were used for downstream analysis for changes in gene expression using DESeq2. MA plot was created from DESeq2 output using R. All raw sequencing files, count matrix used for DESeq2 and metadata information is available at NCBI GEO.

RNA extraction and Quantitative real-time PCR: Total RNA was isolated using Trizol (Invitrogen) in accordance with the manufacturer's instructions followed by Turbo DNA-free DNase (Invitrogen) treatment. RNA was converted into cDNA using Superscript III First Strand synthesis kit (Invitrogen) following manufacturer's instructions. Real-time PCR was performed using Evagreen master mix (Lambda Biotech, St. Louis, Mo.) on a StepOne Plus instrument DNase treatment (Thermo Fisher Scientific). For TERC analysis, Brilliant II 1-step qRT-PCR master mix (Agilent, Santa Clara, Calif.) was used following manufacturer's instructions. Reactions were performed in triplicates with 100 ng of RNA per reaction and used gene-specific (for/rev) primers were as follows:

TERC
(5′-CGCTGTTTTTCTCGCTGACT-3′ (SEQ ID NO. 3),
5′-GCTCTAGAATGAACGGTGGAA-3′(SEQ ID NO. 4)),
TERT
(5′-CGAAAACCTTCCTCAGGACCC-3′ (SEQ ID NO. 5),
5′-GGCCGGCATCTGAACAAAAG-3′ (SEQ ID NO. 6)),
DKC1
(5′-CACTCGCTTGGTGAAGTCACA-3′(SEQ ID NO. 7),
5′-CCGGACAATCCCCACATACT-3′(SEQ ID NO. 8)),
EARN
(5′-CCAGCACAGTAGGAAAGAGAAA-3′(SEQ ID NO. 9),
5′-TCAAGCTCAGTGTCGGAAATC-3′(SEQ ID NO. 10)),
and
ACTB
(5′-CTGTCGAGTCGCGTCCACC-3′(SEQ ID NO. 11),
5′-TCGTCATCCATGGCGAACTGG-3′(SEQ ID NO. 12)).

Detection of telomerase activity: Telomerase activity was assessed by Telomere Repeat Amplification Protocol (TRAP). Briefly, cells were lysed in NP-40 buffer at 4° C. for 20 min and centrifuged at 13,000 rpm for 10 min. Telomere extension reactions were performed using 2, 0.5, and 0.125 μg of protein and following products were amplified by PCR. Adenosine triphosphate, labeled on the gamma phosphate group with 32P (Perkin-Elmer, USA) was used for labelling of PCR products. TRAP was performed in accordance with the manufacturer's instructions (TRAPeze, Millipore Sigma, Burlington, Mass.). Samples were run in 9% polyacrylamide gel in 0.5× TBE buffer for 3 hr at 250V. Gel was dried at 80° C. for 30 min and exposed to Carestream BioMax MR film (Kodak, Rochester, N.Y.).

Telomere length analysis: Telomere length analysis were performed by Telomere Repeat Fragment (TRF) technique. Briefly, DNA was extracted using tail lysis buffer (10 mM Tris-HCl, pH 8.0, 100 mM NaCl, 10 mM EDTA, 0.5% SDS supplemented with 400 μg/ml Proteinase K) at 55° C. overnight. 10 μg of DNA was digested with Rsa1 and Hinf1 restriction enzymes (New England Biolabs, Ispwich, Mass.) overnight and 2.5 μg of product was resolved in a 0.75% agarose gel for 16 hr at 85V in TBE buffer. The gel was incubated with denaturing buffer (1.5M NaCl and 0.5M NaOH) for 45 min followed by neutralizing buffer (1.5M NaCl, 1M Tris-HCl at pH 7.4) for 1 hr. DNA was transferred to a nitrocellulose membrane by capillary action for 2 days in 10× saline-sodium citrate (3M NaCl, 0.3M sodium citrate dehydrate at pH 7.0). After cross-linking, the membrane was hybridized with a ³²P-labelled probe (CCCTAA) and exposed to Carestream BioMax MT film. Mean telomere length was analyzed using TeloTool with the corrected and nonlinear fit order 1 ladder fit options.

Immunoblots. Extraction of protein was performed using NP-40 buffer (25 mM HEPES-KOH, 150 mM KCl, 1.5 mM MgCl2, 10% glycerol, 0.5% NP40, and 5 mM 2ME [pH 7.5]) supplemented with protease and phosphatase inhibitors for 20 minutes on ice and supernatant was then collected. Protein quantification was analyzed by Bradford Assay. Proteins were then resolved in 12% polyacrylamide gels in 1× Tris/glycine/SDS buffer and following resolution was transferred onto nitrocellulose membrane at 400 milliamps for 1:45 hours in 1× Tris/glycine buffer with 20% methanol. Membranes were blocked in 5% milk in TBS buffer and primary γH2AX and PARN (1:1000, and 1:4000, respectively; Abcam, Cambridge Mass.), DKC1 (1:4000, Bethyl Laboratories), Actin (1:2000, Cell Signaling Technologies, Danvers, Mass.) and GAPDH (1:5000; Cell Signaling Technologies) antibodies incubation was performed overnight at 4° C. in 5% BSA in TBS-buffer supplemented with 1% Tween-20 (TBS-T). Secondary antibodies (Li-COR, Lincoln, Nebr.) incubation was in 1% milk in TBS-T for 1 hour. Image capture and signal analysis was performed using odyssey IR scanner and Image Studio software (Li-COR).

Definitive hematopoietic differentiation: Hematopoietic differentiation from hESCs was performed as previously described. Briefly, hESCs were cultured on Matrigel (BD Bioscience) for 1 day to deplete MEFs. On Day 0, cells were dissociated with 0.05% trypsin (Gibco) for 1 min and incubated with serum-free media [SFD: IMDM supplemented with 25% Hams F12, 0.05% BSA, 1× B27 supplement (Gibco), 0.5× N2 supplement (Gibco), 2 mM L-glutamine (Gibco), 50 μg/ml ascorbic acid (Gibco), 4×10⁻⁴ M monothioglycerol (Millipore Sigma), and 150 μg/ml transferrin] supplemented with long/ml BMP4 on 6 well plates coated with a 5% poly(2-hydroxyethyl methacrylate) solution (Millipore Sigma). On Day 1, one additional volume of SFD was added, containing 10 ng/ml BMP4 and 5ng/ml bFGF. On Day 2, media was changed to fresh SFD supplemented with 10 ng/ml BMP4, 1 ng/ml Activin A, 5 ng/ml bFGF, 3 μM CHIR99021, and 604 SB-431542. On Day 3, media was changed to StemPro-34 media [SP-34: StemPro-34 supplemented with 2 mM L-glutamine (Gibco), 1 mM ascorbic acid (Gibco), 4×10⁻⁴M monothioglycerol, 150 μg/ml transferrin] supplemented with 15 ng/ml VEGF and 5 ng/ml bFGF. On Day 6, one additional volume of SP-34 supplemented with 15 ng/ml VEGF, 5 ng/ml bFGF, 20 ng/ml IL-6, 50 ng/ml IGF1, 10 ng/ml IL-11, 200 ng/ml SCF, and 4IU EPO was added. Every step was maintained in a 5% CO2 and 5% O2 incubator. All cytokines were purchased from R&D BioSystems (Minneapolis, Minn.), but EPO and IGF1 were obtained from Peprotech (Rocky Hill, N.J.).

Colony forming culture assays: Colony assays were performed using MethoCult H4034 Optimum (StemCell Technologies, Vancouver, Canada). 10,000 CD34⁺CD43⁻ cells sorted at Day 8 were aggregated overnight in a well of a 96 well low-adhesion plate at 2×10⁵ cells/ml density in 50 μl of SP-34 supplemented with 30 ng/ml TPO, 30 ng/ml IL-3, 100 ng/ml SCF, 10 ng/ml IL-6, 5 ng/ml IL-11, 25 ng/ml IGF1, 2IU EPO, 5 ng/ml VEGF, 5 ng/ml bFGF, 10 ng/ml BMP4, 10 ng/ml FLT3L, and 20 ng/ml SHH. Aggregates were transferred to a well of a Matrigel-coated 24 well plate and 1 ml of the same supplemented SP-34 media was added 6 hr post-transfer. Cells were maintained in 5% CO2 and 5% O2 incubator for 8 days and then placed into 1 ml of MethoCult in which the colonies forming were measured after 12 days. All cytokines were purchased from R&D BioSystems, but EPO and IGF1 were obtained from Peprotech.

Flow cytometry and Cell sorting: Flow cytometry analysis was performed using the BD LSR Fortessa and FACS sorting by BD FACS Aria II at the Department of Pathology & Immunology Flow Cytometry Core at Washington University in St. Louis. The antibodies used are previously described; CD34-APC (clone 8G12), CD34-PE-Cy7 (clone 4H11), CD43-FITC (clone 1G10). All antibodies were purchased from BD Biosciences.

### REFERENCES

1. Savage S. Beginning at the ends: telomeres and human disease. F1000 Res. 2018; 7:7.

2. Wilson D, Link D, Mason P, Bessler M. Inherited bone marrow failure syndromes in adolescents and young adults. Ann Med. 2014; 46(6):353-363.

3. Agarwal S. Evaluation and management of hematopoietic failure in dyskeratosis congenita. Hematol Oncol Clin North Am. 2018; 32(4):669-685.

4. Savage S, Alter B. Dyskeratosis congenita. Hematol Oncol Clin North Am. 2009; 23(2):215-231.

5. Walne A, Dokal I. Advances in the understanding of dyskeratosis congenita. Br J Haematol. 2009; 145(2):164-172.

6. Armanios M, Blackburn E. The telomere syndromes [published correction appears in Nat Rev Genet. 2013; 14(3):235]. Nat Rev Genet. 2012; 13(10): 693-704.

7. Tummala H, Walne A, Collopy L, et al. Poly(A)-specific ribonuclease deficiency impacts telomere biology and causes dyskeratosis congenita. J Clin Invest. 2015; 125(5):2151-2160.

8. Stuart B, Choi J, Zaidi S, et al. Exome sequencing links mutations in PARN and RTEL1 with familial pulmonary fibrosis and telomere shortening. Nat Genet. 2015; 47(5):512-517.

9. Dhanraj S, Gunja S, Deveau A, et al. Bone marrow failure and developmental delay caused by mutations in poly(A)-specific ribonuclease (PARN). J Med Genet. 2015; 52(11):738-748.

10. Stanley S, Gable D, Wagner C, et al. Loss-of-function mutations in the RNA biogenesis factor NAF1 predispose to pulmonary fibrosis-emphysema. Sci Transl Med. 2016; 8(351):351ra107.

11. Mitchell J, Wood E, Collins K. A telomerase component is defective in the human disease dyskeratosis congenita. Nature. 1999; 402(6761):551-555.

12. Heiss N, Knight S, Vulliamy T, et al. X-linked dyskeratosis congenita is caused by mutations in a highly conserved gene with putative nucleolar functions. Nat Genet. 1998; 19(1):32-38.

13. Shukla S, Schmidt J, Goldfarb K, Cech T, Parker R. Inhibition of telomerase RNA decay rescues telomerase deficiency caused by dyskerin or PARN defects. Nat Struct Mol Biol. 2016; 23(4):286-292.

14. Moon D, Segal M, Boyraz B, et al. Poly(A)-specific ribonuclease (PARN) mediates 39-end maturation of the telomerase RNA component. Nat Genet. 2015; 47(12):1482-1488.

15. Boyraz B, Moon D, Segal M, et al. Posttranscriptional manipulation of TERC reverses molecular hallmarks of telomere disease. J Clin Invest. 2016; 126(9):3377-3382.

16. Tseng C, Wang H, Burns A, Schroeder M, Gaspari M, Baumann P. Human telomerase RNA processing and quality control. Cell Rep. 2015; 13(10): 2232-2243.

17. Roake C, Chen L, Chakravarthy A, Ferrell J Jr., Raffa G, Artandi S. Disruption of telomerase RNA maturation kinetics precipitates disease. Mol Cell. 2019; 74(4):688-700.e3.

18. Fok W, Shukla S, Vessoni A, et al. Posttranscriptional modulation of TERC by PAPD5 inhibition rescues hematopoietic development in dyskeratosis congenita. Blood. 2019; 133(12):1308-1312.

19. Mueller H, Wildum S, Luangsay S, et al. A novel orally available small molecule that inhibits hepatitis B virus expression. J Hepatol. 2018; 68(3):412-420.

20. Mueller H, Lopez A, Tropberger P, et al. PAPD5/7 are host factors that are required for hepatitis B virus RNA stabilization. Hepatology. 2019; 69(4): 1398-1411.

21. Fok W, Niero E, Dege C, Brenner K, Sturgeon C, Batista L. p53 Mediates failure of human definitive hematopoiesis in dyskeratosis congenita. Stem Cell Reports. 2017; 9(2):409-418.

22. Venteicher A, Abreu E, Meng Z, et al. A human telomerase holoenzyme protein required for Cajal body localization and telomere synthesis. Science. 2009; 323(5914):644-648.

23. Zhu Y, Tomlinson R, Lukowiak A, Terns R, Terns M. Telomerase RNA accumulates in cajal bodies in human cancer cells. Mol Biol Cell. 2004; 15(1): 81-90.

24. Sturgeon C, Ditadi A, Awong G, Kennedy M, Keller G. Wnt signaling controls the specification of definitive and primitive hematopoiesis from human pluripotent stem cells. Nat Biotechnol. 2014; 32(6):554-561.

25. Winkler T, Hong S, Decker J, et al. Defective telomere elongation and hematopoiesis from telomerase-mutant aplastic anemia iPSCs. J Clin Invest. 2013; 123(5):1952-1963.

26. Donaires F, Alves-Paiva R, Gutierrez-Rodrigues F, et al. Telomere dynamics and hematopoietic differentiation of human DKC1-mutant induced pluripotent stem cells. Stem Cell Res (Amst). 2019; 40:101540.

27. Nagpal N, Wang J, Zeng J, et al. Small-molecule PAPD5 inhibitors restore telomerase activity in patient stem cells [published online ahead of print 21 Apr. 2020]. Cell Stem Cell. doi: 10.1016/j.stem.2020.03.016.

28. Gohring J, Fulcher N, Jacak J, Riha K. TeloTool: a new tool for telomere length measurement from terminal restriction fragment analysis with improved probe intensity correction. Nucleic Acids Res. 2014; 42(3):e21.

29. Ditadi A, Sturgeon CM. Directed differentiation of definitive hemogenic endothelium and hematopoietic progenitors from human pluripotent stem cells. Methods. 2016; 101:65-72.

SEQUENCE LISTING
Amino Acid
PAPD5 protein
Homo Sapiens
SEQ ID NO. 1
MYRSGERLLGSHALPAEQRDFLPLETTNNNNNHHQ
PGAWARRAGSSASSPPSASSSPHPSAAVPAADPAD
SASGSSNKRKRDNKASGGRAAGGGRADGGGVVYSG
TPWKRRNYNQGVVGLHEEISDFYEYMSPRPEEEKM
RMEVVNRIESVIKELWPSADVQIFGSFKTGLYLPT
SDIDLVVFGKWENLPLWTLEEALRKHKVADEDSVK
VLDKATVPIIKLTDSFTEVKVDISFNVQNGVRAAD
LIKDFTKKYPVLPYLVLVLKQFLLQRDLNEVFTGG
IGSYSLFLMAVSFLQLHPREDACIPNTNYGVLLIE
FFELYGRHFNYLKTGIRIKDGGSYVAKDEVQKNML
DGYRPSMLYIEDPLQPGNDVGRSSYGAMQVKQAFD
YAYVVLSHAVSPIAKYYPNNETESILGRIIRVTDE
VATYRDWISKQWGLKNRPEPSCNGPVSSSSATQSS
SSDVDSDATPCKTPKQLLCRPSTGNRVGSQDVSLE
SSQAVGKMQSTQTTNTSNSTNKSQHGSARLFRSSS
KGFQGTTQTSHGSLMTNKQHQGKSNNQYYHGKKRK
HKRDAPLSDLCR
DNA
telomerase (TERC)
Homo Sapiens
SEQ ID NO. 2
GGGTTGCGGAGGGTGGGCCTGGGAGGGGTGGTGGC
CATTTTTTGTCTAACCCTAACTGAGAAGGGCGTAG
GCGCCGTGCTTTTGCTCCCCGCGCGCTGTTTTTCT
CGCTGACTTTCAGCGGGCGGAAAAGCCTCGGCCTG
CCGCCTTCCACCGTTCATTCTAGAGCAAACAAAAA
ATGTCAGCTGCTGGCCCGTTCGCCCCTCCCGGGGA
CCTGCGGCGGGTCGCCTGCCCAGCCCCCGAACCCC
GCCTGGAGGCCGCGGTCGGCCCGGGGCTTCTCCGG
AGGCACCCACTGCCACCGCGAAGAGTTGGGCTCTG
TCAGCCGCGGGTCTCTCGGGGGCGAGGGCGAGGTT
CAGGCCTTTCAGGCCGCAGGAAGAGGAACGGAGCG
AGTCCCCGCGCGCGGCGCGATTCCCTGAGCTGTGG
GACGTGCACCCAGGACTCGGCTCACACATGC

1. A method of treating a telomere-associated disease or disorder in a subject in need thereof, comprising administering a therapeutically effective amount of a PAPD5 inhibitor.

2. The method of claim 1, wherein said step of administering a therapeutically effective amount of a PAPD5 inhibitor comprises the step of administering a therapeutically effective amount of RG7834, or a pharmaceutically acceptable salt thereof

3. The method of claim 2 wherein said therapeutically effective amount of RG7834 rescues human telomerase RNA (TERC) function in the subject.

4. The method of claim 3, wherein said therapeutically effective amount of RG7834 inhibits degradation of TERC in the subject, and/or increases levels of TERC in the subject.

5. (canceled)

6. The method of claim 2, wherein said therapeutically effective amount of RG7834 modulates localization of TERC in the subject.

7. The method of claim 6, wherein modulating localization of TERC in the subject comprises the step of increasing localization of TERC to the cajal bodies of the nucleus of one or more cells of the subject.

8. The method of claim 2, wherein said therapeutically effective amount of RG7834 increases telomere lengths in one or more cells of the subject.

9. The method of claim 8, wherein said in one or more cells of the subject comprises one or more human embryonic stem cells (hESCs) carrying the dyskerin (DKC1) A353V mutation.

10. The method of claim 2, wherein said therapeutically effective amount of RG7834 increases telomere lengths in DKC1 depleted cells of the subject.

11. The method of claim 2, wherein said therapeutically effective amount of RG7834 increases telomere lengths in Poly(A) ribonuclease (PARN) depleted cells of the subject.

12. The method of claim 2, wherein said therapeutically effective amount of RG7834 increases telomerase activity in the subject and/or increases telomerase homeostasis in the subject.

13. (canceled)

14. The method of claim 2, wherein said therapeutically effective amount of RG7834 inhibit DNA damage signaling generated from eroded telomeres in the subject.

15. The method of claim 2, wherein said therapeutically effective amount of RG7834 increases hematopoietic differentiation in one or more cells of the subject.

16. The method of claim 15, wherein said in one or more cells of the subject comprises one or more human embryonic stem cells (hESCs)

17. The method of claim 2, wherein said therapeutically effective amount of RG7834 inhibits 3′ end oligoadenylation of TERC in the subject.

18. (canceled)

19. The method of claim 1, wherein said telomere-associated disease or disorder comprises a telomere-associated disease or disorder selected from the group consisting of: dyskeratosis congenita, aplastic anemia, familial pulmonary fibrosis, and myelodysplastic syndrome.

20-23. (canceled)

24. A method of treating a telomere-associated disease or disorder in a subject in need thereof, comprising administering a therapeutically effective amount of a PAPD5 inhibitor, wherein a therapeutically effective amount of a PAPD5 inhibitor may exhibit one or more of the following:
rescue human telomerase RNA (TERC) function in the subject;
inhibit degradation of TERC in the subject;
increase levels of TERC in the subject;
increase telomerase activity in the subject;
increase telomere homeostasis in the subject;
modulate localization of TERC in the subject;
inhibit DNA damage signaling arising from eroded telomeres in the subject;
increase localization of TERC to the cajal bodies of the nucleus of one or more cells of the subject;
increase telomere length, and preferably increase telomere lengths in human embryonic stem cells (hESCs) carrying the DKC1 A353V mutation;
increase hematopoietic differentiation, and preferably hematopoietic potential in hESCs carrying the DKC1_A353V mutants;
increase telomere lengths in DKC1 depleted cells of the subject;
inhibit 3′ end oligoadenylation of TERC in the subject;
increase telomere lengths in PARN depleted cells of the subject; and
wherein said PAPD5 inhibitor is RG7834, or a pharmaceutically acceptable salt thereof; and

25. (canceled)

26. The method of claim 24, wherein said telomere-associated disease or disorder comprises a telomere-associated disease or disorder selected from the group consisting of: dyskeratosis congenita, aplastic anemia, familial pulmonary fibrosis, and myelodysplastic syndrome.

27. (canceled)

28. A method of preventing a telomere-associated disease or disorder in a subject at risk of developing a telomere-associated disease or disorder, comprising administering a therapeutically effective amount of a PAPD5 inhibitor, wherein a therapeutically effective amount of a PAPD5 inhibitor may prevent one or more of the following indications of said telomere-associate disease or condition:
loss of normal human telomerase RNA component (TERC) function in the subject;
atypical degradation of TERC in the subject;
decreased levels of TERC in the subject;
decreased telomerase activity in the subject;
decreased telomere homeostasis in the subject;
atypical localization of TERC in the subject;
increased DNA damage signaling arising from eroded telomeres in the subject;
decreased hematopoietic differentiation, and preferably hematopoietic potential in hESCs carrying the DKC1_A353V mutants;
decreased localization of TERC to the cajal bodies of the nucleus of one or more cells of the subject;
decreased telomere lengths, and in particular decreased telomere lengths in human embryonic stem cells (hESCs) carrying the DKC1 A353V mutation;
increased 3′ end oligoadenylation of TERC in the subject;
decreased telomere lengths in DKC1 depleted cells of the subject;
decreased telomere lengths in PARN depleted cells of the subject; and
wherein said PAPD5 inhibitor is RG7834, or a pharmaceutically acceptable salt thereof.

29. (canceled)

30. The method of claim 28, wherein said telomere-associated disease or disorder comprises a telomere-associated disease or disorder selected from the group consisting of: dyskeratosis congenita, aplastic anemia, and myelodysplastic syndrome.

31. (canceled)
